(19) United States
(12) Patent Application Publication
(10) Pub. No.: US 2012/0059196 A1
(43) Pub. Date: Mar. 8, 2012

(54) METHOD FOR PREPARING A TERPENYLCYCLOHEXANOL

(75) Inventor: Roland Jacquot, Francheville (FR)

(73) Assignee: RHODIA OPERATIONS, Aubervilliers (FR)

(21) Appl. No.: 13/265,640

(22) PCT Filed: Apr. 21, 2010
(86) PCT No.: PCT/EP10/55245

§ 371 Date: Nov. 16, 2011

(30) Foreign Application Priority Data
Apr. 22, 2009 (FR) 09 01941

(51) Int. Cl. C07C 29/20

(52) U.S. Cl. 568/820

(57) ABSTRACT

A method for preparing a terpenylcyclohexanol using a terpenylphenol is described. A method for preparing a terpenylcyclohexanol by hydrogenation of a terpenylphenol is also described wherein the method includes hydrogenating the latter in a liquid phase, in the presence of a Raney nickel catalyst including residual aluminium and doped with a mixture of iron and chromium.

A subject matter of the invention is a process for the preparation of a terpenylcyclohexanol from a terpenylphenol.

Sandalwood oil is one of the oldest raw materials which, because of its advantageous olfactory properties, has been widely used in perfumery.

However, this natural product is inordinately expensive and synthetic replacement products have been sought after.

One of the first categories proposed is composed of terpenylcyclohexanols. The latter are obtained by condensation of a phenol and camphene in the presence of a catalyst of the Lewis acid type, followed by hydrogenation of the aromatic nucleus in order to result in the cyclohexanol.

Various processes involving reactions of this type are described in the literature.

Mention may in particular be made of U.S. Pat. No. 4,061,686, which describes the condensation of pyrocatechol or 1,2-dihydroxybenzene and camphene in the presence of a Friedel-Crafts catalyst, resulting in an intermediate product which is a mixture of terpenylpyrocatechols, the predominant product of which corresponds to the following formula:

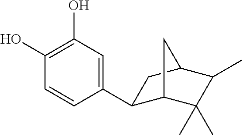

The hydrogenation, using a conventional, Raney nickel catalyst, of the complex mixture obtained results in a sweet-smelling mixture of numerous isomers.

The two main isomers correspond to the following formulae:

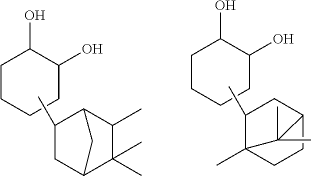

The hydrogenation reaction is carried out at a high temperature of between 200° C. and 300° C., preferably between 225° C. and 250° C., under a hydrogen pressure of between 200 and 250 bar.

The amount of catalyst used represents from 3 to 20% of the weight of the product of the reaction between the catechol and the camphene.

The hydrogenation conditions described in U.S. Pat. No. 4,061,686 are relatively extreme and thus difficult to employ on the industrial scale.

Furthermore, the amount of catalyst to be employed is high since example 9 indicates the use of 30 g of Raney nickel for the hydrogenation of 350 g of substrate.

The Applicant Company intends to provide a process which is more advantageous from an economic viewpoint.

One of the objectives of the present invention is to improve the hydrogenation conditions, in particular to carry out the reaction at a lower temperature.

Another objective of the invention is to employ a smaller amount of catalyst.

There has now been found, and it is this which constitutes the subject matter of the present invention, a process for the preparation of a terpenylcyclohexanol by hydrogenation of a terpenylphenol, characterized in that the hydrogenation of the latter is carried out in the liquid phase in the presence of a catalyst of Raney nickel type which comprises residual aluminum and which is doped with a mixture of iron and chromium.

In accordance with the process of the invention, it has been found that the use of the catalyst as defined according to the invention makes it possible to carry out the hydrogenation reaction at a lower temperature and that the amount of catalyst employed can be reduced without damaging the olfactory quality of the mixture obtained.

The process of the invention applies very particularly to the substrates corresponding to the following general formula:

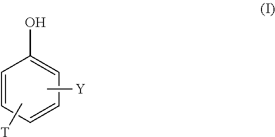

in said formula:
Y representing:
   a hydrogen atom,
   an OH group,
   an OR group in which R represents a linear or branched alkyl group having from 1 to 4 carbon atoms,
T representing a bicyclic terpenyl group comprising 10 carbon atoms.

In accordance with a preferred form of the invention, the terpenylcyclohexanol employed corresponds to the formula (I) in which Y represents an OH group or an OR group in which R represents a methyl or ethyl group.

As regards the terpenyl group T, it represents one of the following groups, alone or as a mixture: bornyl, isobornyl, camphyl, isocamphyl, fenchyl or isofenchyl group.

In fact, the starting terpenylphenol is a mixture of positional isomers and of terpenyl isomers, with the result that the hydrogenated product obtained is also a mixture of isomers.

The term “terpenylphenol” thus refers to the mixture of bornylphenol, isobornylphenol, camphylphenol, isocamphylphenol, fenchylphenol and isofenchylphenol isomers. These terpenyl groups can occur on all the free positions of the aromatic nucleus.

The proportions of the various isomers depend on the nature of the starting substrate and on the conditions of the preparation of the terpenylphenol.

A terpenylphenol which can be prepared according to the various processes described in the literature is involved in the process of the invention.

It concerns a condensation reaction of a phenol and camphene in the presence of a Friedel-Crafts catalyst.

Mention may be made, as examples of catalysts capable of being used, inter alia, of boron trifluoride, a metal halide, such as, for example, aluminum trichloride, ferric chloride or zinc chloride, sulfuric acid, zeolites and clays.

A catalyst suitable for the reaction is boron trifluoride.

Given that the latter is a gas, it is preferable, according to the invention, to resort to boron trifluoride complexes comprising approximately between 20 and 70% by weight of boron trifluoride.

Mention may in particular be made, as examples of complexes, of the complexes comprising boron trifluoride in combination with a solvent chosen from ethyl ether, acetic acid, acetonitrile and, preferably, phenol.

As regards the choice of a zeolite catalyst, recourse is preferably had to a zeolite exhibiting wide pores. Mention may more particularly be made, as preferred examples of zeolites, of zeolites β, zeolites Y and mordenites, all in acid form.

Another catalyst suitable for the condensation reaction is composed of the group of the clays and more particularly the montmorillonites and especially the clays sold by Sud-Chemie, such as the clays K 10 and K 20.

The camphene reacted with the phenol is a commercially available product. Generally, it is a mixture of camphene and tricyclene present at a content of at most 10% of the weight of the mixture and preferably at a content of at most 7%.

The phenolic compound reacting with the camphene preferably corresponds to the following formula:

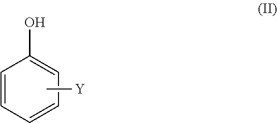

in said formula Y having the meaning given for the formula (I).

The ratio of the number of moles of phenol to the number of moles of camphene varies between 1 and 4 and preferably lies between 1 and 2.

As regards the amount of Lewis acid catalyst, the amount of catalyst depends on the catalyst chosen.

When the catalyst is in the form of a salt or of a complexed salt, the amount thereof employed can vary, for example, between 0.05 and 25 g per mol of phenolic compound and, for a catalyst of clay or zeolite type, the amount is from 0.1 to 1 g per mol of phenolic compound.

The reaction can be carried out in the presence or in the absence of an organic solvent depending on the physical properties of the starting substrate.

The choice of the solvent is such that it must be inert under the reaction conditions of the invention. It must have the property of dissolving the starting phenolic substrate.

Mention may be made, as nonlimiting examples of solvents suitable in this process stage, of aliphatic or of cycloaliphatic hydrocarbons.

Mention may be made, as examples of hydrocarbons, of aliphatic hydrocarbons and more particularly paraffins, such as, in particular, cyclohexane.

As regards halogenated hydrocarbons, mention may more particularly be made of halogenated aliphatic or aromatic hydrocarbons, such as perchlorinated hydrocarbons, for example, in particular, tetrachloromethane, partially chlorinated hydrocarbons, such as dichloromethane, trichloromethane or dichloroethane, monochlorobenzene, 1,2-dichlorobenzene, 1,3-dichlorobenzene, 1,4-dichlorobenzene or mixtures of different chlorobenzenes.

The reaction between the phenol and the camphene is advantageously carried out at a temperature lying between 20° C. and 200° C. and preferably between 20° C. and 180° C.

The process of the invention is generally carried out at atmospheric pressure but pressures slightly greater or lower than atmospheric pressure may also be suitable.

The duration of the reaction can vary, for example, between 2 and 24 hours, preferably between 2 and 12 hours.

At the end of the reaction, the catalyst is removed by a solid/liquid separation technique, preferably by filtration, when the catalyst is heterogeneous or by an aqueous hydrolysis operation, followed by a liquid/liquid separation by settling, when the catalyst is homogeneous.

After removal of the catalyst, the phenolic compound employed in excess is recovered by distillation and can be recycled and the distillation concentrate is subjected to the hydrogenation operation in accordance with the process of the invention.

A catalyst of Raney nickel type is thus involved in the hydrogenation stage.

The Raney nickel conventionally used in reduction reactions and in particular in hydrogenation reactions is a catalyst generally prepared according to the method described below.

A nickel-aluminum alloy is prepared by melting a mixture comprising from 25 to 75% by weight of nickel and from 25 to 75% by weight of aluminum but, generally, a ratio of equal weights is preferred. The melting point is preferably chosen between 1100° C. and 1700° C.

The molten alloy is subsequently solidified, generally in the form of ingots, by casting in molds and cooling to ambient temperature (15 to 25° C.)

In a following operation, the ingots are crushed and milled until the alloy is obtained in the form of a powder.

A basic treatment, which makes possible the dissolution of a portion of the aluminum and thus produces a porous microstructure, is subsequently carried out.

The catalyst obtained is composed of agglomerates of nickel crystallites having a high specific surface and a variable residual aluminum content.

Attack by base is preferably carried out using a concentrated solution of alkali metal hydroxide, preferably sodium hydroxide, (for example 20 to 30% by weight) and an excess of base, the base/alloy, expressed as Al, molar ratio preferably being between 1 and 1.3.

The operation is carried out at a temperature preferably chosen between 50° C. and 100° C.

The catalyst is obtained in the form of a powder in aqueous suspension and it is separated from the aqueous phase, which comprises the alkali metal aluminate.

The catalyst is generally washed in order to remove the excess base.

The doped Raney nickel according to the invention is prepared according to the method of preparation given above with addition of the doping agents iron and chromium to the molten Ni—Al precursor alloy or at the same time as the nickel and aluminum. Metallurgical doping is involved.

The amount of the doping agents employed is such that a catalyst is obtained which exhibits the compositions defined below.

The catalyst involved in the process of the invention advantageously comprises:
from 1 to 5% by weight of chromium
from 1 to 5% by weight of iron
from 5 to 10% by weight of aluminum
from 80 to 93% by weight of nickel.

The catalyst preferably employed in the process of the invention comprises:
from 1.5 to 3% by weight of chromium
from 1 to 4% by weight of iron
from 5 to 7% by weight of aluminum
from 86 to 92.5% by weight of nickel.

The catalyst of the invention having the composition as defined is generally provided in the form of a fine powder having a particle size measured by sieving ranging from 10 to 40 μm.

As the catalyst is a pyrophoric catalyst, it is stored and introduced into the reaction in the form of a basic aqueous suspension having a pH of between 9 and 11 and a concentration variant between 30 and 50% by weight.

In accordance with the process of the invention, the hydrogenation of the terpenylphenol is carried out in the presence of a Raney nickel catalyst as defined.

The amount of hydrogenation catalyst employed, expressed by the ratio of the weight of metal to the weight of compound of formula (I), can vary, for example, between 1 and 10% by weight, preferably between 1 and 5% by weight and more preferably still between 1 and 3% by weight.

The reaction is preferably carried out neat but it is not ruled out to employ an organic solvent when the medium is difficult to handle. Mention may be made, as examples of solvents, of alcohols, such as isopropanol.

The process of the invention is carried out at a temperature chosen within a temperature range extending from 180° C. to 250° C. and more particularly between 190° C. and 220° C.

The reaction takes place under a hydrogen pressure ranging from a pressure slightly greater than atmospheric pressure up to a pressure of several tens of bars. Advantageously, the hydrogen pressure varies between 18 and 30 bar and more preferably between 20 and 25 bar.

At the end of the reaction, a mixture of terpenylcyclohexanol isomers corresponding to the following formula:

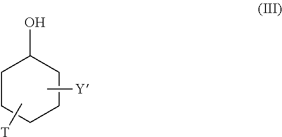

in said formula:
Y′ representing a hydrogen atom when Y represents a hydrogen atom,
Y′ representing a hydrogen atom when Y is an OR group,
Y′ representing an OH group when Y is an OH group,
   is obtained.

When Y is an OR group, an alcoholysis reaction, which corresponds to the formation of an alcohol ROH, also takes place during the hydrogenation operation according to the process of the invention, which alcohol has to be removed during the hydrogenation by continuous or sequential purging operations.

In practice, the process according to the invention can be carried out by introducing, into a stainless steel autoclave, the compound of formula (I), the catalyst and the solvent (water) and then, after the usual purging operations, by feeding the autoclave with an appropriate hydrogen pressure; the contents of the autoclave are subsequently brought with stirring to the appropriate temperature, until absorption ceases. When the consumption of hydrogen ceases, the reactor is purged in order to remove the water and/or the alcohol formed during the hydrogenation reaction. The pressure in the autoclave can be kept constant throughout the duration of the reaction by virtue of successive purging operations which make it possible to remove a light alcohol, if the latter has formed.

At the end of the reaction, the autoclave is cooled and degassed.

The reaction mixture is subsequently treated in a conventional way in order to recover the terpenylcyclohexanol.

To this end, an amount of an organic solvent, preferably an alcohol of low molecular weight, such as, for example, isopropanol, can be added in order to fluidify the reaction mixture.

Subsequently, the catalyst is separated according to conventional solid/liquid separation techniques, preferably by filtration, and the terpenylcyclohexanol is recovered from the filtrate, in particular by distillation.

Implementation examples of the invention, given by way of illustration and without a limiting nature, are given below.

In the examples, the degree of conversion of the terpenylguaiacol is defined as the ratio of the number of moles of terpenylguaiacol converted to the number of moles of terpenylguaiacol charged.

### EXAMPLE 1

### 1. Preparation of the Terpenylguaiacol

751 g of guaiacol and 441 g of molten camphene are successively introduced into a stirred 2 liter stainless steel reactor.

Stirring is carried out and a solution is obtained.

11 g of clay K10 from Sud-Chemie are then added.

The medium immediately turns brown in color and is gradually heated to 150° C.

It is maintained under these conditions for 3 hours.

Under these conditions, none of the camphene or its isomers is any longer detected by gas chromatography (GC).

The temperature is then brought back to 60° C. and the reaction medium is filtered through a bed of Celite (diatomaceous earths) in order to remove the catalyst.

The filtrate is subsequently charged to a 2 liter distillation flask and the excess guaiacol is distilled off at approximately 100° C. under a reduced pressure of 20 mbar of mercury.

682 g of a complex mixture of terpenylguaiacol isomers as defined above are then obtained, namely a mixture of bornyl-, isobornyl-, camphyl-, isocamphyl-, fenchyl- and isofenchylguaiacol isomers.

### 2. Hydrogenation of the Terpenylguaiacol

268 g of terpenylguaiacol and 2.9 g of a catalyst of Raney nickel type comprising 1.6% by weight of chromium, 1.0% by weight of iron and 5.5% by weight of aluminum, the remainder being nickel, are introduced into a 750 ml stainless steel autoclave.

The reactor is then purged with nitrogen and hydrogen.

The reactor is then pressurized under 20 bar of hydrogen, stirring is carried out and heating to 200° C. is carried out gradually.

At this temperature, the headspace of the reactor is purged in order to remove the water introduced with the catalyst of Raney nickel type.

The reactor is again pressurized under 20 bar of hydrogen and the hydrogenation is carried out while keeping the pressure in the reactor constant (20 bar).

Periodically, when the rate of hydrogenation decreases, the hydrogen feed is cut off and the headspace of the reactor is purged in order to remove the methanol and the light products formed in the reaction.

During the reaction, 6 purging operations are carried out and the total duration of hydrogenation is 6 hours.

When it is found, by GC, that all the terpenylguaiacol has reacted, heating is halted and the reactor is purged with nitrogen. Thus, 100% of terpenylguaiacol has been converted.

The temperature is brought back to 60° C. and 50 ml of isopropanol are introduced in order to reduce the viscosity.

The reaction medium is then filtered through Celite in order to remove the catalyst.

After distillation between 135 and 165° C. under a reduced pressure of 2 mbar of mercury, 212 g of a complex mixture of terpenylcyclohexanol isomers (mixture of bornyl-, isobornyl-, camphyl-, isocamphyl-, fenchyl- and isofenchylcyclohexanol isomers) are obtained, which mixture is in accordance with olfactory quality.

The terpenyl unit is not isomerized during the hydrogenation.

### COMPARATIVE EXAMPLE 2

The hydrogenation is carried out as in example 1 but while using the catalyst sold by Degussa BK111W doped with molybdenum and comprising less than 6.5% by weight of aluminum.

On using 2.9 g of this catalyst and after 12 purging operations and hydrogenating for 12 hours, it is found that only 45% of terpenylguaiacol charged has been converted to the expected products.

### COMPARATIVE EXAMPLE 3

The hydrogenation is carried out as in example 1 but while using the catalyst sold by Activated Metal A 5000 comprising 7% of aluminum and 0.16% of iron.

With 2.9 g of this catalyst and after 12 purging operations and hydrogenating for 12 hours, it is found that only 38% of terpenylguaiacol charged has been converted to the expected products.

1. A process for the preparation of a terpenylcyclohexanol, the process comprising hydrogenating the terpenylphenol in a liquid phase with a Raney nickel catalyst which comprises residual aluminum and which is doped with a mixture of iron and chromium.

2. The process as claimed in claim 1, wherein the terpenylphenol corresponds to the following general formula: 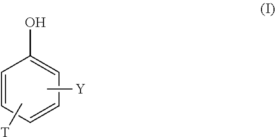
in said formula:
Y representing:
a hydrogen atom,
an OH group,
an OR group in which R represents a linear or branched alkyl group having from 1 to 4 carbon atoms, and
T representing a bicyclic terpenyl group comprising 10 carbon atoms.

3. The process as claimed in claim 2, wherein the terpenylphenol corresponds to the formula (I) in which Y represents an OH group or an OR group in which R represents a methyl or ethyl group.

4. The process as claimed in claim 2, wherein the terpenylphenol corresponds to the formula (I) in which T represents one of the following groups, alone or as a mixture: bornyl, isobornyl, camphyl, isocamphyl, fenchyl or isofenchyl.

5. The process as claimed in claim 2, wherein the terpenylphenol corresponding to the formula (I) is terpenylguaiacol.

6. The process as claimed in claim 1, wherein the Raney nickel catalyst comprises:
from 1% to 5% by weight of chromium
from 1% to 5% by weight of iron
from 5% to 10% by weight of aluminum, and
from 80% to 93% by weight of nickel.

7. The process as claimed in claim 6, wherien the Raney nickel catalyst comprises:
from 1.5% to 3% by weight of chromium
from 1% to 4% by weight of iron
from 5% to 7% by weight of aluminum, and
from 86% to 92.5% by weight of nickel.

8. The process as claimed in claim 1, wherein the catalyst is introduced into the reaction in the form of a basic aqueous suspension having a pH of between 9 and 11 and a concentration variant between 30% and 50% by weight.

9. The process as claimed in claim 1, wherein the amount of hydrogenation catalyst employed, expressed by the ratio of the weight of metal to the weight of compound of formula (I), varies between 1% and 10% by weight.

10. The process as claimed in claim 1, wherein the hydrogenation reaction is carried out at a temperature chosen between 180° C. and 250° C.

11. The process as claimed in claim 1, wherein the hydrogen pressure varies between 18 bar and 30 bar.

12. The process as claimed in claim 1, wherein an alcohol ROH is formed during the hydrogenation of the terpenylphenol corresponding to the formula (I) in which Y represents an OR group and is removed.

13. The process as claimed in claim 9, wherein the amount of hydrogenation catalyst employed varies between 1% and 5% by weight.

14. The process as claimed in claim 9, wherein the amount of hydrogenation catalyst employed varies between 1% and 3% by weight.

15. The process as claimed in claim 10, wherein the hydrogenation reaction is carried out at a temperature chosen between 190° C. and 220° C.

16. The process as claimed in claim 11, wherein the hydrogen pressure varies between 20 bar and 25 bar.
